(19) United States
(12) Patent Application Publication
(10) Pub. No.: US 2025/0316349 A1
(43) Pub. Date: Oct. 9, 2025

(54) System and Method for Identity Matching

(71) Applicant: Elevance Health, Inc., Indianapolis, IN (US)

(72) Inventors: Leela Prasad Peripydi, Indianapolis, IN (US); Venkat Kambalapally, Indianapolis, IN (US)

(21) Appl. No.: 19/170,938

(22) Filed: Apr. 4, 2025

Related U.S. Application Data
(60) Provisional application No. 63/575,355, filed on Apr. 5, 2024.

(51) Int. Cl. G16H 10/60; G06F 16/2457

(52) U.S. Cl. CPC G16H 10/60; G06F 16/24578

(57) ABSTRACT

A method of determining whether a first record and a second record are a match may include performing probabilistic matching, including assigning weights to record attributes to create weighted attributes and computing a probabilistic matching score using the weighted attributes, and performing rule based deterministic matching. The method may also include, returning a result that indicates a match based on the probabilistic matching score and the rule based deterministic matching; performing a modeled analysis of the first record and the second record based on a combined result of the rule based deterministic matching and the probabilistic matching score; returning a result that indicates a match based on a determination, via the modeled analysis, that the first record and the second record are a match; and returning a result that indicates that manual review is needed based on an inconclusive result via the modeled analysis.

### TECHNICAL FIELD

The disclosed implementations relate generally to data integrity, and specifically to detecting duplicative data records that relate to a single subject matter.

### BACKGROUND

In the healthcare industry, especially on the health insurance side, different medical records are constantly generated each with their own independent patient name. Identifying a unique individual from a set of these medical records by matching demographic information may be necessary, to avoid both false conflation of medical records of two patients, and/or incomplete medical records for any given patient. The information that can be collected is limited and sparse. Regulations and business practices limit the ability to place mandatory policies or “must have” attributes in patient records. Competition amongst providers creates an incentive between providers to refuse to disclose all patient information to one another, e.g., when a patient changes insurers. Existing exchange protocols standardizes member data exchanges between providers, but the data required by the protocols may be incomplete, which may result from mutual desires amongst competitors to not share information with one another. These reasons make the membership records sparsely populated with useful information.

For example, patient records for different patients within a household may result in false match detections, as information in the records for the patients will be same due to systematic reasons, which may include family names, addresses, and phone numbers all matching. False positive matches such as these may also result from data copying, e.g., when the patient records are created. These reasons make the correct matching of records very difficult.

Other industries have faced similar “record linkage” problems. However, in other industries, collaborative co-ordinations of the participants, and/or pressure from governments, have enabled those industries to find a solution for this problem with solutions such as unique identifiers (e.g., ID numbers) and/or industry wide mandates. In the healthcare industry, particularly in the United States, due to confusion on ownership of member records and information generated based on services provided, existing approaches are not sufficient to avoid over-matching and under-matching.

Existing matching systems try to create estimated models with the available information on the member records. Because of sparsity of the data, results provided by existing models will not represent the real world. This deviation can go in both direction, over-matching—false positive and under-matching—false negative. A false positive may result in two real-world patients having their medical records merged with one another. Negative results of this may include violations of both patients' medical privacy expectations, and difficulty in providing medical care because the medical record contains erroneous information with respect to one of the patients. A false negative may result in one real-world patient having two separate medical records associated with him or her. Negative results from false negatives may include impact to quality of care caused by incomplete information in each of the medical records. This may also result in incorrect risk assessment, and duplicative communication between the insurer and the patient.

Accordingly, a system that can minimizes over-matches and under-matches would improve patient care, customer service, and regulatory compliance. Identifying an individual among a set of records with minimum number of false matches (over and under) and have processes to improve matching accuracy over time would be advantageous.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method of determining whether a first record and a second record are a match because they relate to a single subject. The method of determining also includes performing probabilistic matching between the first record and the second record, the probabilistic matching may include i) receiving a plurality of record attributes of each of the first record and the second record, ii) assigning a plurality of weights to the plurality of record attributes of each of the first record and the second record to create a plurality of weighted attributes, iii) computing a probabilistic matching score for each of the record attributes using the plurality of weighted attributes. The determining also includes determining whether the probabilistic matching indicates that the first record and the second record are a match. The determining also includes performing rules—based deterministic matching between the first record and the second record, the rule based deterministic matching may include applying a plurality of matching rules to the first record and the second record, each of the plurality of matching rules relating to a specified attribute of the first record and the same specified attribute of the second record, that do not match, where the rule indicates that a difference in the specified attribute likely does not indicate a non-match. The determining also includes upon a determination that (i) the probabilistic matching score indicates that the first record and the second record are a match, and (ii) the rule based deterministic matching indicates that the first record and the second record are a match, returning a result that indicates a match. The determining also includes upon a determination that a combined result of the rule based deterministic matching and the probabilistic matching score returns an inconclusive result, performing a modeled analysis of the first record and the second record, the modeled analysis may include using computer based intelligence using a machine learning model to compare the first record and the second record to determine whether the first record and the second record are definitively a match, are definitively not a match, or their match status is inconclusive. The determining also includes upon a determination via the modeled analysis machine learning model, that the first record and the second record are a match, returning the result that indicates a match. The determining also includes upon an inconclusive result via the modeled analysis, returning a result that indicates that manual review is needed. Other embodiments of this aspect include corresponding computer systems, apparatus, and computer programs recorded on one or more computer storage devices, each configured to perform the actions of the methods.

Implementations may include one or more of the following features. The method where each of the plurality of record attributes is represented by a token, and where the probabilistic matching further may include computing a probabilistic matching score for each of the tokens. The machine learning model is trained on historical data relating to previous pairs of records and a determined match status relating to each of the previous pairs of records. The machine learning model receives, as inputs, a pair of data records and a match indication, and where the machine learning model trains a classifier associated with the machine model based on the inputs. The pair of data records received as an input may include a pair of siblings with similar sounding names. The rules based deterministic matching is performed by a rules based deterministic module, and where the rules based deterministic module may include a false negatives classifier, a false positives engine, and a recertification module. The rules based deterministic matching is performed using rules relating to newborn patients. Implementations of the described techniques may include hardware, a method or process, or computer software on a computer-accessible medium.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is an overview of a system that may be used to perform identity matching in accordance with one embodiment of the present disclosure.

FIG. 2 is a block diagram of a system to perform identify matching in accordance with one embodiment of the present disclosure.

FIG. 3 is a process diagram of an information flow for use in identity matching in accordance with one embodiment of the present disclosure.

FIG. 4 is a flow diagram representing a process for identity matching in accordance with one embodiment of the present disclosure.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

While different approaches to record linkage have been evaluated, most of them result in sub-optimal matching results, due to at least the following reasons in the Healthcare industry. Health care enrollment data collection is sparse. is no National level patient IDs do not exist, and insurers are not permitted to require patients to provide other national unique identifiers such as social security numbers. In some instances, health care services need to be provided even before the member enrolls into the system, which results in creation of health care records that later need to be matched to a patient record that may be a duplicate of another record, and/or may be a false match for another record.

Records matching may be attempted using probabilistic matching, deterministic rule-based matching, machine learning, artificial intelligence, reference data supported matching, and data stewardship. The methods individually method can in some instances provide up to 98% accuracy. A false match rate of 2%, when spread across a large sample of records, such as all insureds covered by a health insurer, results in many false matches, which cause inefficiencies and other problems as noted above. However, when refinement is attempted, to increase accuracy, these methods become very complicated and accuracy trends to deteriorate over time. Accordingly, improving accuracy, to identity an individual among a set of records with minimum number of false matches (over and under) is desirable. Processes to improve matching accuracy over time is similarly desirable.

Accordingly, a multi step process that uses the existing options of matching with data stewardship is disclosed. The process increases accuracy from 98% maximum to 99.998%, and then accuracy may be further improved with a supporting system (e.g., a data stewardship processes) that becomes less onerous when the machine process is more accurate. Steps as disclosed herein may be integrated, each step may use information collected or generated in predecessor steps. Each step follows its own methodology to acquire as much matching accuracy as possible, and also shares that information to next steps.

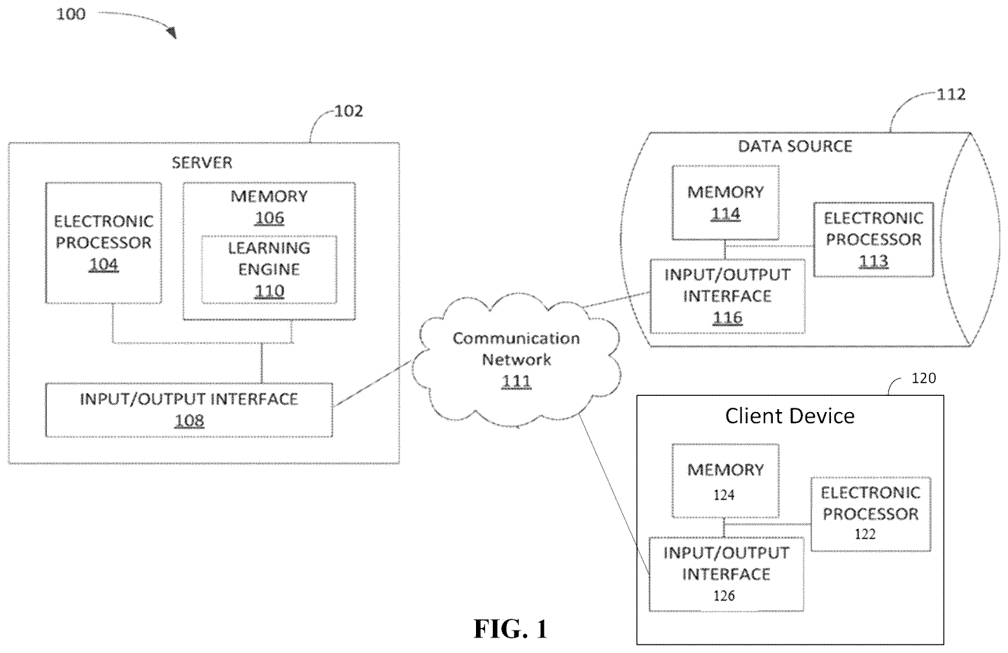

FIG. 1 illustrates a system 100 for detecting and repairing record linkage issues, including false positive and false negative detection, according to some embodiments of the invention. The system 100 includes a server 102 that includes a plurality of electrical and electronic components that provide power, operational control, and protection of the components within the server 102. For example, as illustrated in FIG. 1, the server 102 may include an electronic processor 104 (e.g., a microprocessor, application-specific integrated circuit (ASIC), or another suitable electronic device), a memory 106 (e.g., a non-transitory, computer-readable storage medium), and an input/output interface 108. The electronic processor 104, the memory 106, and the input/output interface 108 communicate over one or more connections or buses. The server 102 illustrated in FIG. 1 represents one example of a server and embodiments described herein may include a server with additional, fewer, or different components than the server 102 illustrated in FIG. 1. Also, in some embodiments, the server 102 performs functionality in addition to the functionality described herein. Similarly, the functionality performed by the server 102 (i.e., through execution of instructions by the electronic processor 104) may be distributed among multiple servers. Accordingly, functionality described herein as being performed by the electronic processor 104 may be performed by one or more electronic processors included in the server 102, external to the server 102, or a combination thereof.

The memory 106 may include read-only memory (“ROM”), random access memory (“RAM”) (e.g., dynamic RAM (“DRAM”), synchronous DRAM (“SDRAM”), and the like), electrically erasable programmable read-only memory (“EEPROM”), flash memory, a hard disk, a secure digital (“SD”) card, other suitable memory devices, or a combination thereof. The electronic processor 104 executes computer-readable instructions (“software”) stored in the memory 106. The software may include firmware, one or more applications, program data, filters, rules, one or more program modules, and other executable instructions. For example, the software may include instructions and associated data for performing the methods described herein. For example, as illustrated in FIG. 1, the memory 106 may store a learning engine (e.g., “software”) 110 for performing one or more of the functions described herein, which may include probabilistic matching, deterministic matching, machine learning, artificial intelligence, or the like. However, in other embodiments, the functionality described herein as being performed by the learning engine 110 may be performed through one or more software modules stored in the memory 106 or external memory.

The input/output interface 108 allows the server 102 to communicate with devices external to the server 102. For example, as illustrated in FIG. 1, the server 102 may communicate with one or more data sources 112 through the input/output interface 108. In particular, the input/output interface 108 may include a port for receiving a wired connection to an external device (e.g., a universal serial bus (“USB”) cable and the like), a transceiver for establishing a wireless connection to an external device (e.g., over one or more communication networks 111, such as the Internet, a local area network (“LAN”), a wide area network (“WAN”), and the like), or a combination thereof.

In some embodiments, the server 102 also receives input from one or more peripheral devices, such as a keyboard, a pointing device (e.g., a mouse), buttons on a touch screen, a scroll ball, mechanical buttons, and the like through the input/output interface 108. Similarly, in some embodiments, the server 102 provides output to one or more peripheral devices, such as a display device (e.g., a liquid crystal display (“LCD”), a touch screen, and the like), a printer, a speaker, and the like through the input/output interface 108. In some embodiments, output may be provided within a graphical user interface (“GUI”) (e.g., generated by the electronic processor 104 executing instructions and data stored in the memory 106 and presented on a touch screen or other display) that enables a user to interact with the server 102. In other embodiments, a user may interact with the server 102 through one or more intermediary devices, such as a personal computing device laptop, desktop, tablet, smart phone, smart watch or other wearable, smart television, and the like). For example, a user may configure functionality performed by the server 102 as described herein by providing data to an intermediary device that communicates with the server 102. In particular, a user may use a browser application executed by an intermediary device to access a web page that receives input from and provides output to the user for configuring the functionality performed by the server 102.

As illustrated in FIG. 1, the system 100 includes one or more data sources 112. Each data source 112 may include a plurality of electrical and electronic components that provide power, operational control, and protection of the components within the data source 112. In some embodiments, each data source 112 represents a server, a database, a personal computing device, or a combination thereof. For example, as illustrated in FIG. 1, each data source 112 may include an electronic processor 113 (e.g., a microprocessor, ASIC, or other suitable electronic device), a memory 114 (e.g., a non-transitory, computer-readable storage medium), and an input/output interface 116. The data sources 112 illustrated in FIG. 1 represents one example of data sources and embodiments described herein may include a data source with additional, fewer, or different components than the data sources 112 illustrated in FIG. 1. Also, in some embodiments, the server 102 communicates with more or fewer data sources 112 than illustrated in FIG. 1.

The input/output interface 116 allows the data source 112 to communicate with external devices, such as the server 102. For example, as illustrated in FIG. 1, the input/output interface 116 may include a transceiver for establishing a wireless connection to the server 102 or other devices through the communication network 111 described above. Alternatively, or in addition, the input/output interface 116 may include a port for receiving a wired connection to the server 102 or other devices. Furthermore, in some embodiments, the data sources 112 also communicate with one or more peripheral devices through the input/output interface 116 for receiving input from a user, providing output to a user, or a combination thereof. In other embodiments, one or more of the data sources 112 may communicate with the server 102 through one or more intermediary devices. Also, in some embodiments, one or more of the data sources 112 may be included in the server 102.

The memory 114 of each data source 112 may store patient data and the like. For example, the data sources 112 may include an electronic medical record (“EMR”) database, a claims database, a patient database, and the like. In some embodiments, as noted above, data stored in the data sources 112 or a portion thereof may be stored locally on the server 102 (e.g., in the memory 106).

User device 120 may also be connected to communication network 111, for communication with server 102 and/or with data source 112. Inputs and outputs 118 may flow between server 102, e.g., via input/output interface 108, and user device 120, e.g., via input/output interface 126. Inputs may include pairs of records to be checked for matches and “record linkages” as described herein. Outputs may include match determinations via probabilistic matching, deterministic matching, and/or machine learning, as described in more detail below.

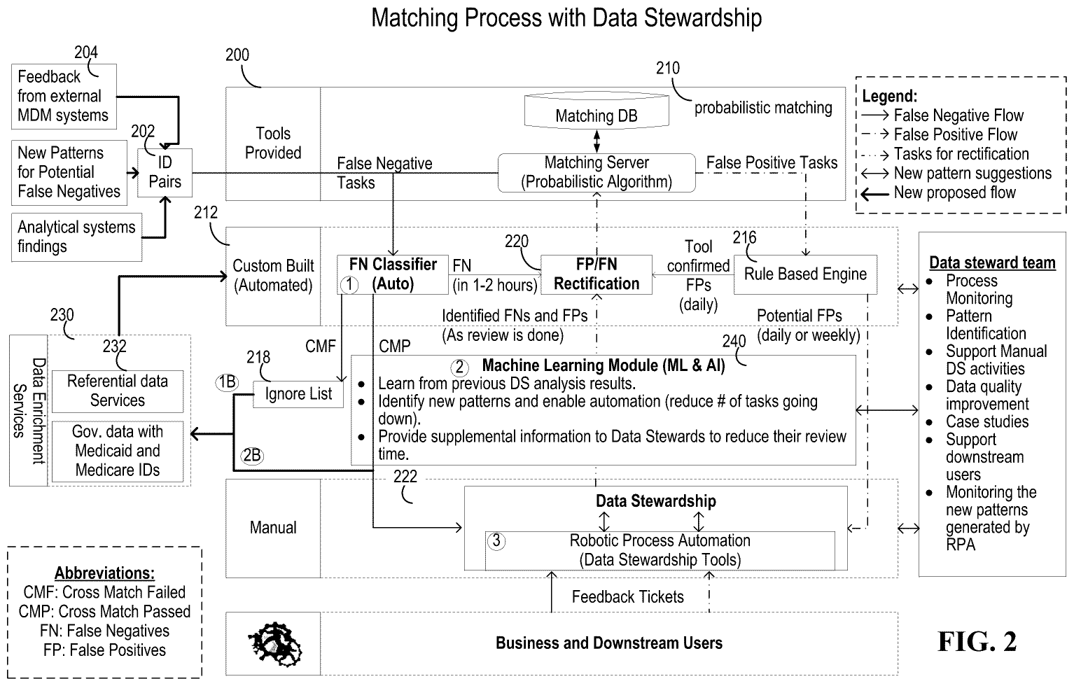

FIG. 2 is a block diagram of a system in accordance with one aspect of the present disclosure. System 200 as shown in FIG. 2 is a system for matching pairs of accounts to determine whether they relate to the same patient. The system receives a pair of Medical IDs (“MCIDS”) 202 from a pair of medical records to determine whether they relate to the same patient. MCIDs may be sourced from BCBSA with MMI ID 204. MDM_ID potential anomaly suggestion can come from external MDM system that are working with same data set 204. Another set of potential anomaly suggestion can come from previously identified patterns from experience 206. Another set of potential anomaly suggestion can come from downstream analytical systems that review MDM_IDs with other information from life cycle of the member 208.

MCID pairs may then be fed to a probabilistic matching module 210. Probabilistic matching module 210 may be configured to calculate a probability that two records belong to the same patient, based on the probability of two records with certain attributes in common, are the same patient, even when other attributes may not match. Probabilistic matching module 210 may be implemented as a Matching server, such as server 102 of FIG. 1. In some embodiments, matching module 210, e.g., via a matching server, collects a data set, generates needed meta data for matching, performs the matching, and takes decisions on assigning ID. Once the decision is finalized, information relating to the decision is persisted into the Matching DB for future reuse. Information relating to the decision may include original information, metadata and the final decision.

Probabilistic matching module 210 may compare tokens of different attributes in the membership record to determine the likelihood of a match. A token may be a data structure designed to represent an attribute of a patient record, such as the patient name, address, age, etc. In some embodiments, tokens may be alphanumeric representations of data, generally excluding separators such as spaces.

Probabilistic matching module 210 may calculate a weighted average of the values of the tokens. Probabilistic matching module 210 may perform the calculation based on pre-defined weightages of the attribute. Pre-defined weights may be determined based on historical information relating to previous false positives, false negatives, or matches between records having the same attribute in common or not having that attribute in common. The weighted average represents the matching scope for given two records. For example, patient name might have a higher likelihood of indicating a match than a patient address, as multiple discrete patients, e.g., family members, roommates, etc., may be more likely to live at the same address, but may be less likely to have the same name, which does happen (e.g., amongst parents and children) but not as often. Therefore, patient name may be weighted higher than patient address when performing probabilistic matching. Different types of matching, and different weights of different attributes, may all be implemented as part of an MDM Algorithm, which may create a probabilistic matching score.

Probabilistic matching module 210 may determine whether the probabilistic matching score satisfies certain matching score criteria, which may include threshold scores. Different next step actions may be taken by the system upon the pair of records, depending on whether the probabilistic matching score reaches or exceeds defined thresholds. For example, if the probabilistic matching score is above a score threshold, which may be referred to as an Auto-Matching Threshold, then this means a match is detected and can be considered confirmed without running the pair of records through the remaining aspects of system 200. However, if the score is below the Auto-Matching Threshold, but above a lower Manual Review Threshold, then system will then send the records to rules based engine 212.

Probabilistic matching module 210 may perform probabilistic matching evaluations on certain fields associated with a record, which may include phonetic, nick name match, frequency based matching evaluation, edit distance match, anonymous values, and partial matching. Phonetic matching may evaluate names that are spelled differently to determine if they might be pronounced the same. Nick name match may evaluate two records with different first names to determine whether one is a known nick name for the other. Distance matching may evaluate to determine a distance between two addresses, e.g., home addresses, listed in two records. Anonymous values may evaluate whether any of the fields reflects intentional concealment, e.g., on the part of the patient when filling out a form that led to the creation of or update to the record.

Use cases for probabilistic matching module 210 may also include matching individuals who may have moved to different ZIP code, handling of “N/A” or similarly uninformative fields, identifying patients who might be the same person despite having different last names, which may results from, e.g., a marriage or a separation, analysis of MCIDs with large numbers of matched records, transposed first and last names, parents and children who may have both the same first name and the same last name, twins who may have the same last name, contact information, and birth date, siblings who may have similar sounding or spelled first names, or spouses with copied demographic information, which, e.g., may be incorrect for one of the spouses.

As noted above, if the probabilistic matching score of a pair of records is between the Auto-Matching Threshold and the Manual Review ThresholdManual Review Threshold then the records will be sent to one or more rules based engines which are represented as a class as rules based engine 212, which may also be performed at server 102, which may occur after server 102 receives input from user device 116. In some embodiments, false negative tasks, which may include pairs with potential for a false negative, may be sent to false negatives classifier 214, which may be a rules based engine with rules designed to identify false negatives as potential matches. False positive tasks, which may include pairs of records with potential for false positive may be sent to a false positives rules based engine 216 with rules designed to filter out false positives. In some embodiments, potential false negatives may be identified via a “potential match score” of the pair being between a set of thresholds. Other that methods may also be used to identify Potential False positive and potential False Negatives.

In False Negatives classifier 214 and false positives rules based engine 216, a set of deterministic rules are utilized to confirm the match or to keep records separated. Deterministic rules are rules that will always produce the same output from the same input. These rules are also used to determine whether a task needs to be created for the data steward to review and take custom decision, which will be explained further below. Deterministic rules help to classify the tasks generated by previous steps and decide whether manual review is needed, or auto decision can be implemented. We support trigger-based rule execution and also scheduled execution depending on the scope.

Deterministic rules that may be applied by false negatives classifier 214 and/or by false positives rules based engine 216 may include matching individuals who moved across ZIP codes, handline “NA” or similarly obviously incorrect data in fields such as first name, or identifying last name changes resulting from marriage and/or separation. Deterministic rules may also include values in fields within a record that are designed to preserve, or assist in potentially preserving, anonymity. Deterministic rules may also include analysis of MCIDs with large numbers of matched records. Deterministic rules may also include a rule to match records where a first name in one record is the last name in the other, and vice versa. Deterministic rules may also be used to identify two records with many overlapping fields resulting from parents (e.g., fathers) having the same first and last names as their children (e.g., sons). Deterministic rules may also include patients who entered the system as newborns, who have anonymous first and/or last names because they were treated with medical care before they were named. Other deterministic rules may relate to handling of potential overlays caused by systems that were a source of the records in question. Deterministic rules may also include rules for resolving generated false negative tasks.

In some embodiments, some records that pass through false negatives classifier 214 may be classified as “Cross Match Failed” as a result of the deterministic rules applied to the record pair by false negatives classifier 214. Record pairs that are classified as Cross Match Failed may be added to ignore list 218. Ignore list 218 may be passed to data enhancement services 230, which may include referential data services 232. Data enhancement services 230 may also include government data, which may include Medicaid and/or Medicare identifiers, e.g., identification numbers.

Other records that pass through false negatives classifier 214 may be categorized as “Cross Match Passed” as a result of the deterministic rules applied to the record pair by false negatives classifier 214. Cross Match Passed records may then be passed to data stewardship 222. Other records that pass through false negatives classifier 214 may be categorized as false negatives. False negatives may be passed to a false positives/false negatives recertification module 220, which will be discussed below.

False Positive tasks that are run through false positives rules based engine 216 may be confirmed as false positives, or may be categorized as potential false positives. Confirmed false positives may be passed to false positives/false negatives recertification module 220. Potential false positives may also be sent to data stewardship 222.

After receiving false negatives and confirmed false positives, false positives/false negatives recertification module 220 may then 220: receive confirmation basee on the type of the task. Received confirmation may be or include “Potential False Positive,” which is really a false positive or it is not. If it is false positive, existing records that are associated with MDM-ID may then be split into new MDM_IDs. If it is not a false positive, data may be created and/or saved to indicate that the review occurred, a problem was not found, and the task is resolved.

Received confirmation may also include “Potential False Negative,” which is whether the pair is really a false negative or it is not. If the task is a false negative then all the records belonging to multiple MDM-ID in the task are brought together, e.g., merged, into the same MDM_ID, after which, only one MDM_ID will survive. If the task is not a false negative data may be created and/or saved to indicate that the review occurred, a problem was not found, and the task is resolved.

In other embodiments, deterministic rules such as those represented by false negatives classifier 214 and/or false positives rules based engine 216 may be employed before probabilistic matching module 210, rather than afterwards as depicted in FIG. 1. In such embodiments, deterministic rules may be applied to identify potential matches and definite non-matches. Potential matches may then be sent to probabilistic matching module for scoring. In other embodiments, a result of an application of one or more deterministic rules may be saved as associated with the pair of records, and all pairs of records will go through both the probabilistic matching module and one or more deterministic rules based engine, after which the result may be evaluated in conjunction with a probabilistic matching score before.

Cross Match Passed results from false negatives classifier 214, and potential false positives passed from false positives rules based engine 216 may then be transmitted to data stewardship 222. Data stewardship 222 may include review by a team of data stewards led by a lead data stewards review tasks that are assigned for manual review. Review may be enabled via a User Interface (“UI”) which may be web based. The UI may also enable a data steward to see information that was collected from source systems, metadata generated by previous steps, e.g., metadata generated using different methods, at one place to take an empowered decision on the given task.

In some aspects, each task will go through 2 individual data stewards (let's call them A and B) review, independently. If the final decisions of these Data stewards match, then action is taken to rectify the task. If there is a conflict in the decisions taken by data stewards, then an opportunity is provided for them to discussion, exchange their viewpoints and come to a single decision. If they can not agree to a single decision, then task will be reviewed with the Data Stewardship-Lead and a final decision is taken. Over the time, all the knowledge collected is consolidated into a Playbook.

Data stewardship 222 may include process monitoring, pattern identification, data quality improvement, and case studies. Data stewardship 222 may also include support for downstream users. Results of the data stewardship process 222 may then be sent to machine learning module 240. Machine learning module 240 may use various techniques, including machine learning and artificial intelligence, to improve the process of data linkage. Machine learning may be used to enhance decision-making, which may be trained by a training corpus created by the results of the probabilistic matching, the deterministic rules based matching, and/or the data stewardship process. In some embodiments, the order may vary. In some embodiments, machine learning module 240 may be used after false positives/false negatives analysis, and before data stewardship. In other embodiments, data stewardship review may occur before machine learning module 240 is actuated. In other embodiments, data stewardship may be periodic throughout. In some embodiments, fewer than all steps may be involved, as an earlier step in the process may result in a definitive answer, making subsequent steps unnecessary for that particular pair of records.

Different ML& AI modules can be added to take decisions based on the previous decisions taken by data stewards. Machine learning may also be used to classify and rectify the tasks, e.g., the false positive tasks and the false negative tasks, that were generated by the probabilistic data matching and/or the deterministic rules based matching. Machine learning may also be used to predict anomalies based on previous experience, which may be helpful in flagging the most important cases for stewardship review. Machine learning and/or artificial intelligence may include handling of anonymous values in fields, handling of twins who may be at risk for false positives because they share many fields, and task resolution for false negatives. ML and AI may be used to detect and match patterns in what kinds of records may present false negatives and/or false positives. Detected patterns may be used to inform future iterations of the selection process for data. ML and AI may include Gen AI, Deep learning, classifier, auto learning, or supervised learning models. Other types of models may also be used.

Various machine learning techniques may be used to train and operate models to perform various steps described herein. Models may be trained and operated according to various machine learning techniques. Such techniques may include, for example, neural networks (such as deep neural networks and/or recurrent neural networks), inference engines, trained classifiers, etc. Examples of trained classifiers include Support Vector Machines (SVMs), neural networks, decision trees, AdaBoost (short for “Adaptive Boosting”) combined with decision trees, and random forests. Focusing on SVM as an example, SVM is a supervised learning model with associated learning algorithms that analyze data and recognize patterns in the data, and which are commonly used for classification and regression analysis. Given a set of training examples, each marked as belonging to one of two categories, an SVM training algorithm builds a model that assigns new examples into one category or the other, making it a non-probabilistic binary linear classifier. More complex SVM models may be built with the training set identifying more than two categories, with the SVM determining which category is most similar to input data. An SVM model may be mapped so that the examples of the separate categories are divided by clear gaps. New examples are then mapped into that same space and predicted to belong to a category based on which side of the gaps they fall on. Classifiers may issue a “score” indicating which category the data most closely matches. The score may provide an indication of how closely the data matches the category.

In order to apply the machine learning techniques, the machine learning processes themselves need to be trained. Training a machine learning component such as, in this case, one of the first or second models, requires establishing a “ground truth” for the training examples. In machine learning, the term “ground truth” refers to the accuracy of a training set's classification for supervised learning techniques. Various techniques may be used to train the models including backpropagation, statistical learning, supervised learning, semi-supervised learning, stochastic learning, or other known techniques.

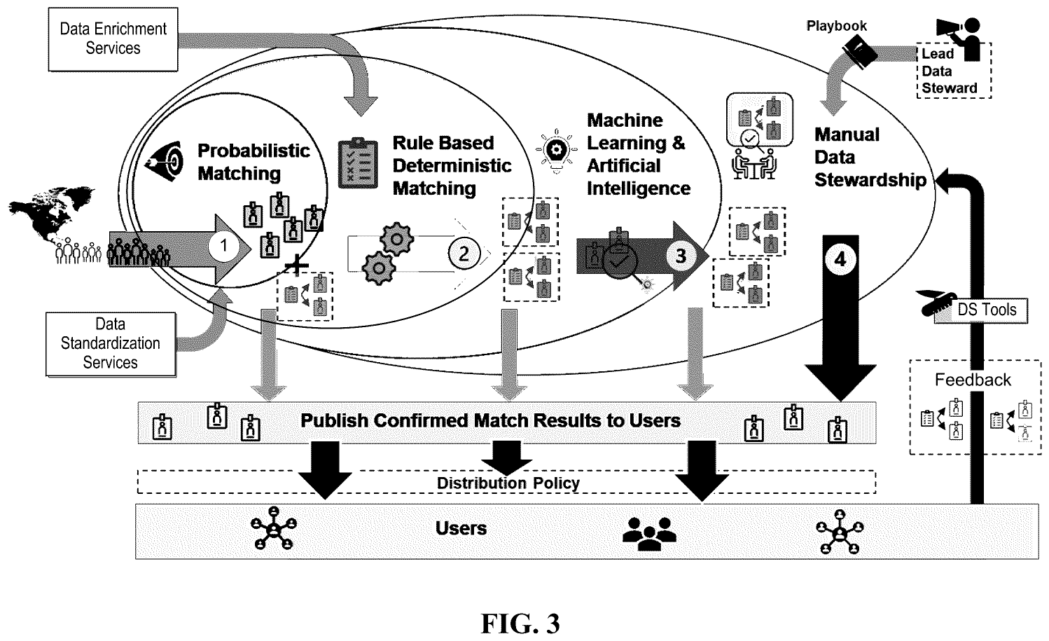

FIG. 3 is a process flow diagram in accordance with one aspect of the present disclosure. Process 300 is a flow of the operation of system 200 as disclosed in and described with reference to FIG. 2. Process 300 begins with probabilistic matching step 302, which may be performed by probabilistic matching module 210 of system 200. As discussed in more detail above, probabilistic matching may include calculating weighted values for different attributes of patient records to determine a weighted probability of a match based on the pair of records having certain attributes in common and other attributes not in common. Probabilistic matching may also include data standardization services that may be used to determine whether the same attribute in two records should be treated as the same, despite minor differences between them.

Depending on a probabilistic matching score that may be generated by probabilistic matching step 302, results of probabilistic matching step 302 may then be transmitted to rule based deterministic matching step 304, which may be performed by rules based engine section 212, which may include False Negatives classifier 214 and false positives rules based engine 216. As discussed in more detail above, deterministic rules may be applied by deterministic matching step 304 to confirm or reject false negatives and false positives.

The order in which steps are performed may vary. In some embodiments, rules based matching step 304 may occur prior to probabilistic matching step 302. In some embodiments, the steps may be executed in the order shown in FIG. 3. In other embodiments, information in a subsequent step may be fed back, e.g., via a feedback loop, to previously executed components, e.g., to improve future executions of the earlier modules. This may occur, in some embodiments, after a decision has been reached about a pair of records.

Combined results of probabilistic matching step 302 and rules based matching step 304 may then be published to users or otherwise distributed 306, if a confidence level of the combined matching status of probabilistic matching step 302 and rules based matching step 304 is sufficient. In some embodiments, the meta data generated in previous steps may be vectorized and send to machine learning module 240.

If the confidence level is not sufficient, the pairs of records may be passed to machine learning and artificial intelligence step 308 for further analysis. Machine learning and artificial intelligence analysis may be performed by machine learning module 240. As discussed above machine learning and artificial intelligence step 308 may include trained machine learning models and algorithms that use historical data to relating to previous true matches, false positives, or false negatives, to predict whether a pair of records may be a true match or a false positive. Machine learning and artificial intelligence step 308 may reach a sufficient confidence level of a match or a non-match for a given pair of records, and the result may therefore be published to users or otherwise distributed 306. Certain pairs of records may then be further evaluated by data stewardship step 310. The system may then return a result, either that the two records are a match, or that the two records are not a match, or that the match status of the two records could not be conclusively determined and further evaluation is required. Further evaluation may also be informed by data generated by the process, which may be published in a readable or reviewable form to guide the evaluation.

Artifacts may also be used in the various steps in the process, as described in and with reference to FIG. 3. For example, probabilistic matching step 302 may use a probabilistic matching algorithm that may be stored as an artifact. Rule based deterministic matching step 304 may use a rule book artifact. Machine learning/Artificial Intelligence step 308 may use one or more machine learning models, which may be stored as artifacts. In some embodiments, artifacts may be compressed folders with sets of files required by a particular step. In some embodiments, artifacts may be managed via a web service.

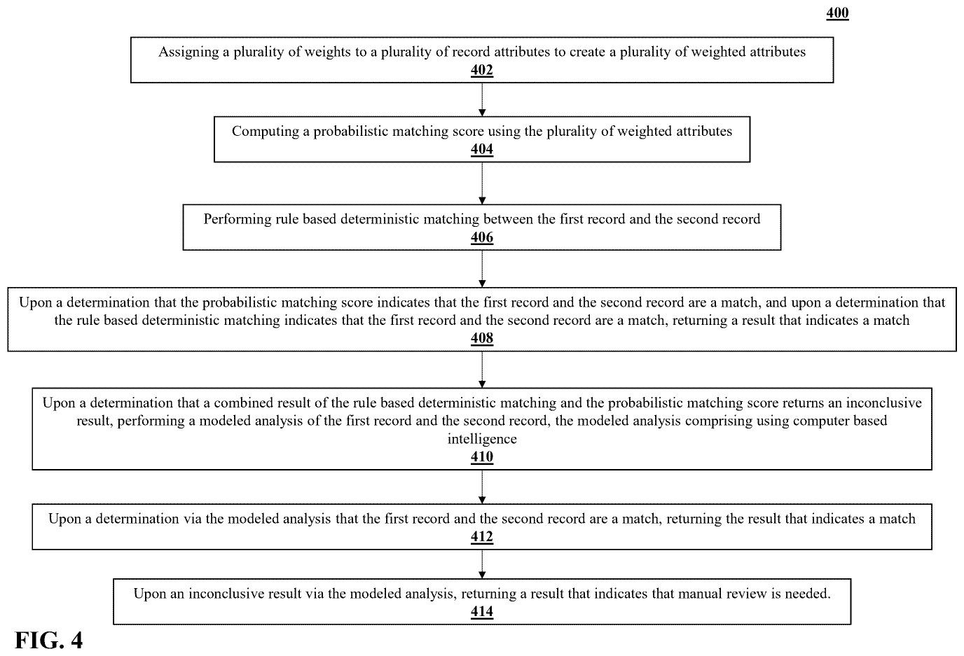

Turning now to FIG. 4, a flow diagram of a process 400 for a method of determining whether a first record and a second record are a match because they relate to a single subject is shown. Probabilistic matching is performed, e.g., by probabilistic matching module 210, between the first record and the second record. The probabilistic matching may include assigning (402) a plurality of weights to a plurality of record attributes to create a plurality of weighted attributes and computing (404) a probabilistic matching score using the plurality of weighted attributes. Rule based deterministic matching between the first record and the second record may then be performed (406), e.g., by rules based engine 212, which may, in some embodiments, include false negatives classifier 214 and/or false positives rules based engine 216. The rule based deterministic matching includes applying a plurality of matching rules to the first record and the second record, each of the plurality of matching rules relating to a specified attribute of the first record and the same specified attribute of the second record, which do not match, wherein the rule indicates that a difference in the specified attribute likely does not indicate a non-match.

Upon a determination that the probabilistic matching score indicates that the first record and the second record are a match, and upon a determination that the rule based deterministic matching indicates that the first record and the second record are a match, a result that indicates a match may be returned (408). Upon a determination that a combined result of the rule based deterministic matching and the probabilistic matching score returns an inconclusive result, a modeled analysis of the first record and the second record may be performed (410), e.g., by machine learning module 240 of FIG. 2. The modeled analysis may include using computer based intelligence.

Upon a determination, via the modeled analysis, that the first record and the second record are a match, a result that indicates a match may be returned (412), which may include publishing such a result as in step 306 of FIG. 3. Finally, upon an inconclusive result via the modeled analysis, a result that indicates that manual review is needed may be returned (412).

The system disclosed herein improves upon prior record linkage techniques by combining methodologies and using the results of a first methodology to inform a second, and, in some embodiments, a third. The system disclosed herein, and variations thereof, may also improve the separation of Artifacts from the process, which enables flexibility and adaption of ever evolving business requirements. The present disclosure may also be used to deduplicate other kinds of records other than patient records. Other kinds of records that may deduplicated may include provider records, product records, or other data elements. The present disclosures may also be used for intercompany matching of records, or reconciliation of records, e.g., with non-standard attributes.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

What is claimed is:

1. A method of determining whether a first record and a second record are a match because they relate to a single subject, comprising:
performing probabilistic matching between the first record and the second record, the probabilistic matching including:
i) receiving a plurality of record attributes of each of the first record and the second record;
ii) assigning a plurality of weights to the plurality of record attributes of each of the first record and the second record to create a plurality of weighted attributes;
iii) computing a probabilistic matching score between the first record and the second record as a function of each of the record attributes and each of the plurality of weighted attributes;
determining whether the probabilistic matching indicates that the first record and the second record are a match;
performing rule-based deterministic matching between the first record and the second record, the rule based deterministic matching comprising applying a plurality of matching rules to the first record and the second record, each of the plurality of matching rules relating to a respective attribute of the first record and the same respective attribute of the second record to determine whether there is a match between the respective attributes of the first record and the second record;
upon a determination that (i) the probabilistic matching score indicates that the first record and the second record are a match, and (ii) the rule based deterministic matching indicates that the first record and the second record are a match, returning a result that indicates a match;
upon a determination that a combined result of the rule based deterministic matching and the probabilistic matching score returns an inconclusive result, using a machine learning model to compare one or more attributes of the first record and the second record to determine whether the first record and the second record are a match, are not a match, or their match status is inconclusive;
upon a determination, via the machine learning model, that the first record and the second record are a match, returning the result that indicates a match; and
upon an inconclusive result via the modeled analysis, returning a result that indicates that manual review is needed.

2. The method of claim 1, wherein each of the plurality of record attributes is represented by a token, and wherein the probabilistic matching further comprises computing a probabilistic matching score for each of the tokens.

3. The method of claim 1, wherein the machine learning model is trained on historical data relating to previous pairs of records and a determined match status relating to each of the previous pairs of records.

4. The method of claim 3, wherein the machine learning model receives, as inputs, a pair of data records and a match indication, and wherein the machine learning model trains a classifier associated with the machine model based on the inputs.

5. The method of claim 4, wherein the pair of data records received as an input comprises a pair of siblings with similar sounding names.

6. The method of claim 1, wherein the rules based deterministic matching is performed by a rules based deterministic module, and wherein the rules based deterministic module comprises a false negatives classifier, a false positives engine, and a recertification module.

7. The method of claim 1, wherein the rules based deterministic matching is performed using rules relating to newborn patients.

8. A system for determining whether a first record and a second record are a match because they relate to a single subject, comprising a processor and a memory, the memory containing computer executable instructions that, when executed by the processor, instruct the processor to:
perform probabilistic matching between the first record and the second record, the probabilistic matching including:
i) receiving a plurality of record attributes of each of the first record and the second record;
ii) assigning a plurality of weights to the plurality of record attributes of each of the first record and the second record to create a plurality of weighted attributes;
iii) computing a probabilistic matching score between the first record and the second record as a function of each of the record attributes and each of the plurality of weighted attributes;
determine whether the probabilistic matching indicates that the first record and the second record are a match;
perform rule-based deterministic matching between the first record and the second record, the rule based deterministic matching comprising applying a plurality of matching rules to the first record and the second record, each of the plurality of matching rules relating to a respective attribute of the first record and the same respective attribute of the second record to determine whether there is a match between the respective attributes of the first record and the second record;
upon a determination that (i) the probabilistic matching score indicates that the first record and the second record are a match, and (ii) the rule based deterministic matching indicates that the first record and the second record are a match, return a result that indicates a match;
upon a determination that a combined result of the rule based deterministic matching and the probabilistic matching score returns an inconclusive result, use a machine learning model to compare one or more attributes of the first record and the second record to determine whether the first record and the second record are a match, are not a match, or their match status is inconclusive;
upon a determination, via the machine learning model, that the first record and the second record are a match, return the result that indicates a match; and
upon an inconclusive result via the modeled analysis, return a result that indicates that manual review is needed.

9. The system of claim 8, wherein each of the plurality of record attributes is represented by a token, and wherein the probabilistic matching further comprises computing a probabilistic matching score for each of the tokens.

10. The system of claim 8, wherein the machine learning model is trained on historical data relating to previous pairs of records and a determined match status relating to each of the previous pairs of records.

11. The system of claim 10, wherein the machine learning model receives, as inputs, a pair of data records and a match indication, and wherein the machine learning model trains a classifier associated with the machine model based on the inputs.

12. The system of claim 11, wherein the pair of data records received as an input comprises a pair of siblings with similar sounding names.

13. The system of claim 8, wherein the rules based deterministic matching is performed by a rules based deterministic module, and wherein the rules based deterministic module comprises a false negatives classifier, a false positives engine, and a recertification module.

14. The system of claim 8, wherein the rules based deterministic matching is performed using rules relating to newborn patients.
